(19) United States
(12) Patent Application Publication
(10) Pub. No.: US 2020/0185067 A1
(43) Pub. Date: Jun. 11, 2020

(54) PATIENT INFORMATION PROVIDING SYSTEM, PATIENT INFORMATION PROVIDING METHOD, AND PROGRAM

(71) Applicant: OPTiM CORPORATION, Saga-shi (JP)

(72) Inventor: Shunji SUGAYA, Tokyo (JP)

(21) Appl. No.: 16/309,952

(22) PCT Filed: Jun. 16, 2016
(86) PCT No.: PCT/JP2016/067965

§ 371 Date: Jan. 21, 2019

(51) Int. Cl. G16H 10/60; G06K 7/14; G16H 15/00

(52) U.S. Cl. CPC G16H 10/60; G16H 15/00; G06K 7/1417

(57) ABSTRACT

An object of the present invention is to provide a patient information providing system, a patient information providing method, and a program capable of acquiring and displaying patient information of a patient without inputting identification information. A patient information providing system 1, which accesses a storage unit that stores patient information to acquire and display patient information, acquires a captured image captured by an information terminal 100 or a two-dimensional code read by the information terminal 100, specifies an ID of a patient appearing in the acquired captured image or the ID of a patient associated with the two-dimensional code acquired by the information terminal 100, acquires patient information associated with the specified ID of the patient from the storage unit, and displays the acquired patient information on a display unit.

### TECHNICAL FIELD

The present invention relates to a patient information providing system, a patient information providing method, and a program for accessing a storage unit storing patient information to acquire and display the patient information.

### BACKGROUND ART

Conventionally, when acquiring patient information, which is various information related to a patient, in a medical field, a medical staff inputs a patient's name, his/her identifier, a password, or the like into a terminal, thereby accessing patient information which is stored in a storage unit or the like of a server to acquire the patient information and display the acquired patient information on the terminal.

As a configuration for displaying such patient information, a configuration in which the patient information is acquired from the server or the like in which an electronic medical record is stored and the acquired patient information is displayed on the terminal is disclosed (see Patent Document 1).

### PRIOR ART DOCUMENT

### Patent Document

Patent Document 1: Japanese Patent Application Publication No. 2015-153148

### SUMMARY OF THE INVENTION

### Technical Problem

However, in the configuration of Patent Document 1, it is necessary to input identification information such as the patient's name and the identifier of the medical staff. In addition, there is also a problem that the acquired patient information is not necessarily the patient information of the patient desired by the medical staff.

It is an object of the present invention to provide a patient information providing system, a patient information providing method, and a program capable of acquiring and displaying patient information of a patient without inputting identification information.

### Technical Solution

The present invention provides the following solutions.

An invention according to a first aspect provides a patient information providing system for accessing a storage unit that stores patient information to acquire and display the patient information, the patient information providing system including a captured image acquiring unit that acquires a captured image captured by a terminal, a patient specifying unit that specifies an ID of a patient appearing in the acquired captured image by image recognition, a patient information acquiring unit that acquires patient information associated with the specified ID of the patient from the storage unit, and a display unit that displays the acquired patient information on a display unit.

According to the first aspect of the invention, a patient information providing system for accessing a storage unit that stores patient information to acquire and display the patient information acquires a captured image captured by a terminal, specifies an ID of a patient appearing in the acquired captured image by image recognition, acquires patient information associated with the specified ID of the patient from the storage unit, displays the acquired patient information on a display unit.

The invention according to the first aspect is a category of the patient information providing system, but even in other categories such as a method or a program, exhibits the same action and effect corresponding to the category.

An invention according to a second aspect provides a patient information providing system for accessing a storage unit that stores patient information to acquire and display the patient information, the patient information providing system including a two-dimensional code acquiring unit that acquires a two-dimensional code read by a terminal, a patient specifying unit that specifies an ID of a patient associated with the acquired two-dimensional code, a patient information acquiring unit that acquires patient information associated with the specified ID of the patient from the storage unit, and a display unit that displays the acquired patient information on a display unit.

According to the second aspect of the invention, a patient information providing system for accessing a storage unit that stores patient information to acquire and display the patient information acquires a two-dimensional code read by a terminal, specifies an ID of a patient associated with the acquired two-dimensional code, acquires patient information associated with the specified ID of the patient from the storage unit, and displays the acquired patient information on a display unit.

The invention according to the second aspect is a category of the patient information providing system, but even in other categories such as a method or a program, exhibits the same action and effect corresponding to the category.

An invention according to a third aspect provides a patient information providing system which is the invention according to the first or second aspect, wherein the display unit displays a change in the acquired patient information as a graph.

According to the third aspect of the invention, a patient information providing system, which is the invention according to the first or second aspect, displays a change in the acquired patient information as a graph.

An invention according to a fourth aspect provides a patient information providing system, which is the invention according to the first aspect, further including an image capturing position information acquiring unit that acquires position information of a location where the captured image is captured by the terminal, and wherein the display unit displays the acquired patient information in association with the acquired position information of the captured image.

According to the fourth aspect of the invention, a patient information providing system, which is the invention according to the first aspect, acquires position information of a location where the captured image is captured by the terminal, and displays the acquired patient information in association with the acquired position information of the captured image.

An invention according to a fifth aspect provides a patient information providing system, which is the invention according to the second aspect, further including a reading position information acquiring unit that acquires position information of a location where the two-dimensional code is read by the terminal, and wherein the display unit displays the acquired patient information in association with the acquired position information of the two-dimensional code.

According to the fifth aspect of the invention, a patient information providing system, which is the invention according to the second aspect, acquires position information of a location where the two-dimensional code is read by the terminal, and displays the acquired patient information in association with the acquired position information of the two-dimensional code.

An invention according to a sixth aspect provides a patient information providing system, which is the invention according to the first aspect, further including an evidence utilization unit that uses the captured image as an evidence of the specified patient when accessing the storage unit.

According to the sixth aspect of the invention, a patient information providing system, which is the invention according to the first aspect, uses the captured image as an evidence of the specified patient when accessing the storage unit.

An invention according to a seventh aspect provides a patient information providing system, which is the invention according to the second aspect, further including an evidence utilization unit that uses the two-dimensional code as an evidence of the specified patient when accessing the storage unit.

According to the seventh aspect of the invention, a patient information providing system, which is the invention according to the second aspect, uses the two-dimensional code as an evidence of the specified patient when accessing the storage unit.

An invention according to an eight aspect provides a patient information providing method of accessing a storage unit that stores patient information to acquire and display the patient information, the patient information providing method including acquiring a captured image captured by a terminal, specifying an ID of a patient appearing in the acquired captured image by image recognition, acquiring patient information associated with the specified ID of the patient from the storage unit, and displaying the acquired patient information on a display unit.

An invention according to a ninth aspect provides a patient information providing method of accessing a storage unit that stores patient information to acquire and display the patient information, the patient information providing method including acquiring a two-dimensional code read by a terminal, specifying an ID of a patient associated with the acquired two-dimensional code, acquiring patient information associated with the specified ID of the patient from the storage unit, and displaying the acquired patient information on a display unit.

An invention according to a tenth aspect provides a program for causing a patient information providing system accessing a storage unit that stores patient information to acquire and display the patient information to execute acquiring a captured image captured by a terminal, specifying an ID of a patient appearing in the acquired captured image by image recognition, acquiring patient information associated with the specified ID of the patient from the storage unit, and displaying the acquired patient information on a display unit.

An invention according to an eleventh aspect provides a program for causing a patient information providing system accessing a storage unit that stores patient information to acquire and display the patient information to execute acquiring a two-dimensional code read by a terminal, specifying an ID of a patient associated with the acquired two-dimensional code, acquiring patient information associated with the specified ID of the patient from the storage unit, and displaying the acquired patient information on a display unit.

### Effects of the Invention

According to the present invention, it is possible to provide a patient information providing system, a patient information providing method, and a program capable of acquiring and displaying patient information of a patient without inputting identification information.

### BRIEF DESCRIPTION OF THE DRAWINGS

FIG. 1 is a diagram showing an overview of a patient information providing system 1.

FIG. 2 is an overall configuration diagram of a patient information providing system 1.

FIG. 3 is a functional block diagram of a server 10 and an information terminal 100.

FIG. 4 is a diagram showing a patient ID database creating process executed by a server 10 and an information terminal 100.

FIG. 5 is a diagram showing an image specifying process executed by a server 10 and an information terminal 100.

FIG. 6 is a diagram showing an image specifying process executed by a server 10 and an information terminal 100.

FIG. 7 is a diagram showing a code reading process executed by a server 10 and an information terminal 100.

FIG. 8 is a diagram showing a code reading process executed by a server 10 and an information terminal 100.

FIG. 9 is a diagram showing an example of patient information displayed by an information terminal 100.

### DETAILED DESCRIPTION OF THE EMBODIMENTS

Hereinafter, embodiments for carrying out the present invention are described with reference to the drawings. It is to be understood that the embodiments are merely examples and the scope of the present invention is not limited to the disclosed embodiments.

Overview of Patient Information Providing System 1

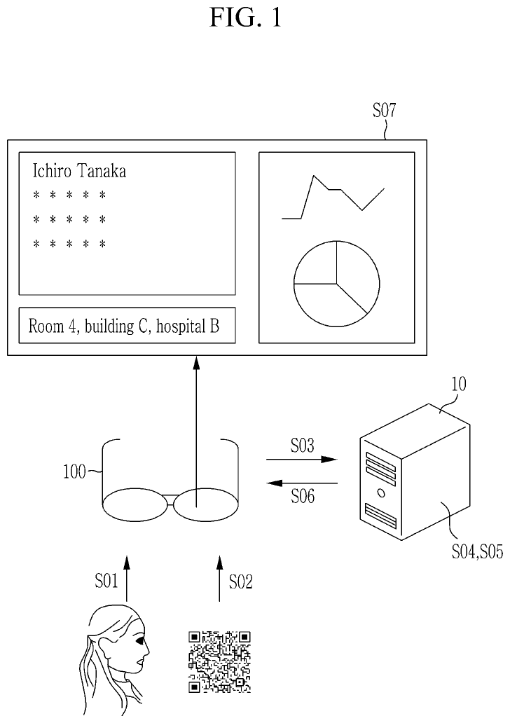

An overview of a preferred embodiment of the present invention is described with reference to FIG. 1. FIG. 1 is a diagram for explaining an overview of a patient information providing system 1 which is a preferred embodiment of the present invention. The patient information providing system 1 includes a server 10 and an information terminal 100.

In FIG. 1, the number of information terminal(s) 100 is not limited to one, but may be two or more. Further, the server 10 or the information terminal 100 is not limited to an existing device, but may be a virtual device. Furthermore, each process to be described below may be realized by either the server 10 or the information terminal 100, or both of them.

The server 10 is a server device capable of performing data communication with the information terminal 100. The server 10 stores patient information, which is various information on a patient, in its own storage unit, and accesses the storage unit to acquire patient information and display the patient information on the information terminal 100.

The information terminal 100 is a terminal device capable of performing data communication with the server 10 and operated by a medical staff. The information terminal 100 is, for example, a mobile phone, a portable information terminal, a tablet terminal, a personal computer, an electric appliance such as a netbook terminal, a slate terminal, an electronic book terminal, or a portable music player, a wearable terminal such as a smart glasses or a head mount display, or other goods. In the following description, it is assumed that the information terminal 100 is the wearable terminal.

The information terminal 100 captures a captured image of a target patient (step S01). In addition, the information terminal 100 reads an information code such as a two-dimensional code or a one-dimensional code of the target patient (step S02). The processing in either step S01 or step S02 or in both step S01 and step S02 may be executed. The information terminal 100 executes capturing the patient or reading the information code by an imaging capturing device such as a camera owned by the information terminal 100. The information terminal 100 captures a face, an upper body, a whole body, or the like of the patient. The information terminal 100 reads an information code attached to clothing or the body of the patient.

Further, the information terminal 100 may be configured to acquire its own position information and store the position information in its own storage unit at the time of executing the processing of step S01 or step S02. The information terminal 100 uses its own position information stored at this time in processing to be described below. Furthermore, the information terminal 100 may execute capturing the patient or reading the information code by a configuration other than the image capturing device.

The information terminal 100 transmits captured image information indicating data of the captured image or code information indicating data of the information code to the server 10 (step S03).

The server 10 receives the captured image information or the code information, and specifies an ID of the target patient based on the received captured image information or code information (step S04). The server 10 performs image analysis on the captured image based on the captured image information, thereby extracting a feature amount of the patient appearing in the captured image. The server 10 specifies the ID associated with the extracted feature amount of the patient, based on the extracted feature amount of the patient and a patient ID database in which feature amounts of patients and IDs are associated with each other in advance. In addition, the server 10 reads information stored in the information code based on the code information and specifies an ID of the patient associated with the stored information.

In a case where the ID of the patient is stored in the code information, the server 10 may be configured to specify the ID of the patient stored in the code information.

The server 10 acquires patient information associated with the specified patient ID (step S05). The server 10 extracts patient information associated with the specified patient ID based on the specified patient ID and a patient information database in which patient IDs and patient information are associated with each other in advance. The patient information is, for example, various information relating to a patient to be preserved as an electronic medical record, such as a name, an age, an occupation, a sex, an insurer number, an injury/illness name, a visit start date, a hospital stay period, a past medical history, a medication content, a surgical content, or a remark.

Further, the server 10 may be configured to use the captured image information or the code information as an evidence for identification of the patient. The server 10 may permit access to the patient information database if the captured image or information code represents the patient himself/herself, and deny the access to the patient information database if it does not represent the patient himself/herself.

The server 10 transmits the acquired patient information to the information terminal 100 (step S06).

The information terminal 100 receives the patient information and displays the patient information on a display unit of the information terminal 100 (step S07). Further, the information terminal 100 may be configured to display a change in the patient information as a graph. Furthermore, the information terminal 100 may be configured to display the patient information in association with the stored position information.

The above is the overview of the patient information providing system 1.

System Configuration of Patient Information Providing System 1

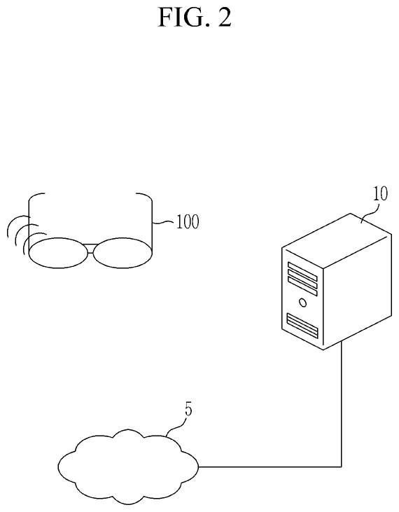

A system configuration of a patient information providing system 1 which is a preferred embodiment of the present invention is described with reference to FIG. 2. FIG. 2 is a diagram showing a system configuration of a patient information providing system 1 which is a preferred embodiment of the present invention. The patient information providing system 1 includes a server 10, an information terminal 100, and a public line network (Internet network, third or fourth generation communication network, or the like) 5. In addition, the number of information terminal(s) 100 is not limited to one, and may be two or more. Further, the server 10 and the information terminal 100 are not limited to existing devices and may be virtual devices. Furthermore, each process to be described below may be realized by either the server 10 or the information terminal 100, or both of them.

The server 10 is the above-described server device having functions to be described below.

The information terminal 100 is the above-described terminal device having functions to be described below. In the following description, it is assumed that the information terminal 100 is a wearable terminal.

Description of Functions

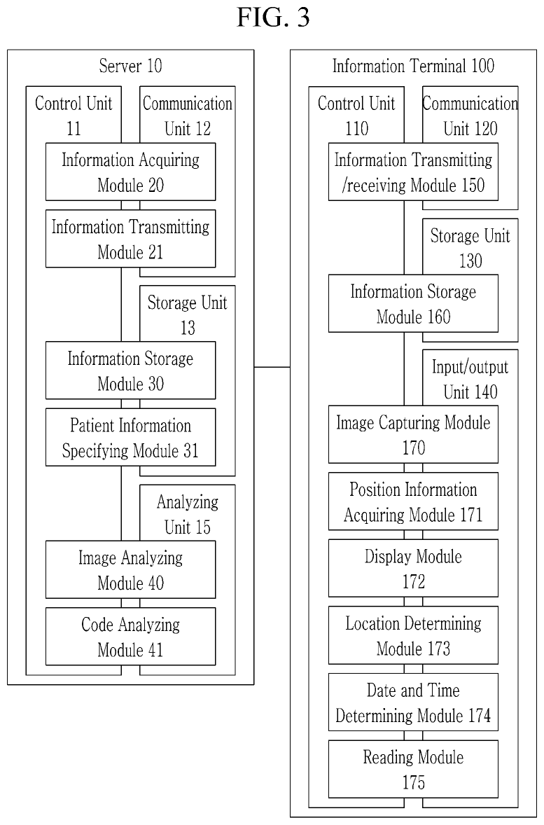

Functions of a patient information providing system 1 which is a preferred embodiment of the present invention are described with reference to FIG. 3. FIG. 3 is a functional block diagram of a server 10 and an information terminal 100.

The server 10 includes, as a control unit 11, a CPU (Central Processing Unit), a RAM (Random Access Memory), a ROM (Read Only Memory), and the like, and includes, as the communication unit 12, a device for enabling communication with other devices, for example, a WiFi (Wireless Fidelity) compliant device conforming to IEEE 802.11. Further, the server 10 includes, as a storage unit 13, a data storage unit such as a hard disk, a semiconductor memory, a recording medium, a memory card, or the like. The storage unit 13 stores various databases to be described below. Furthermore, the server 10 includes, as an analyzing unit 15, an image analyzing device which executes image analysis of a captured image, an information code analyzing device which analyzes various types of information embedded in an information code, or the like.

In the server 10, the control unit 11 reads a predetermined program, thereby realizing an information acquiring module 20 and an information transmitting module 21 in cooperation with the communication unit 12. Further, in the server 10, the control unit 11 reads a predetermined program, thereby realizing an information storage module 30 and a patient information specifying module 31 in cooperation with the storage unit 13. Furthermore, in the server 10, the control unit 11 reads a predetermined program, thereby realizing an image analyzing module 40 and a code analyzing module 41 in cooperation with the analyzing unit 15.

Like the server 10, the information terminal 100 includes, as a control unit 110, a CPU, a RAM, a ROM, and the like, includes, as a communication unit 120, a WiFi compatible device for enabling communication with other devices or a telephone device for calling a phone number, or the like, and includes, as a storage unit 130, a data storage unit. Further, the information terminal 100 includes, as an input/output unit 140, a display unit which outputs and displays data or images controlled by the control unit 110, an input unit, such as a touch panel, a keyboard, or a mouse, which accepts an input from a medical staff, an image capturing device which captures a captured image of a patient, a code reading device which reads an information code, a position information acquiring device which acquires its own position information from a GPS or the like, or the like.

In the information terminal 100, the control unit 110 reads a predetermined program, thereby realizing an information transmitting/receiving module 150 in cooperation with the communication unit 120. Further, in the information terminal 100, the control unit 110 reads a predetermined program, realizing an information storage module 160 in cooperation with the storage unit 130. Furthermore, in the information terminal 100, the control unit 110 reads a predetermined program, thereby realizing an image capturing module 170, a position information acquiring module 171, a display module 172, a location determining module 173, a date and time determining module 174, and a reading module 175 in cooperation with the input/output unit 140.

Patient ID Database Creating Process

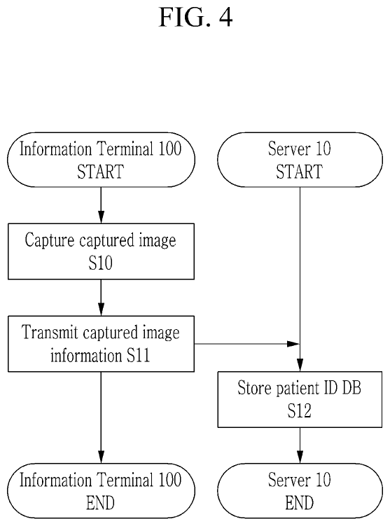

A patient ID database creating process executed by a patient information providing system 1 is described with reference to FIG. 4. FIG. 4 is a flowchart showing a patient ID database creating process executed by a server 10 and an information terminal 100. The processing executed by the above-described modules of each device is described together with this processing.

An image capturing module 170 captures a captured image of a patient (step S10). In step S10, the image capturing module 170 captures a captured image capable of identifying a patient such as a face, upper body, or whole body of the patient. The processing in step S10 is executed, for example, at the timing when the patient visits a hospital for the first time, such as at the time of the first medical examination. Further, the image capturing module 170 may be configured to capture other parts. In this case, the image capturing module 170 preferably captures a part capable of identifying the patient. Furthermore, the timing of capturing the patient may be other timing.

An information transmitting/receiving module 150 transmits captured image information which is data of the captured image to the server 10 (step S11). In step S11, the information transmitting/receiving module 150 assigns an ID of the patient to the captured image information, and transmits the ID to the server 10.

An information acquiring module 20 receives the captured image information and the ID. An information storage module 30 creates and stores a patient ID database in which the captured image information and the ID are associated with each other (step S12).

The above is the patient ID database creating process.

Image Specifying Process

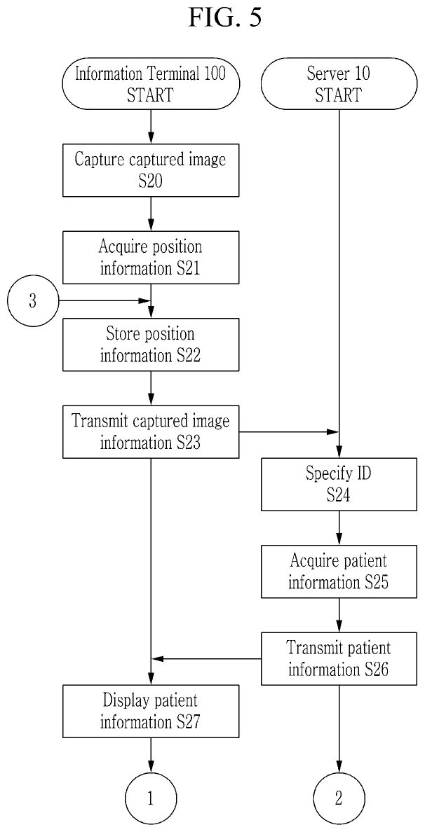

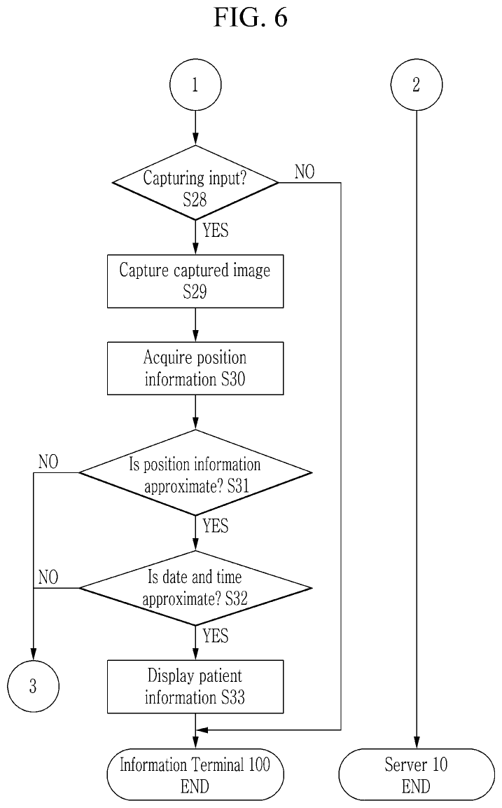

An image specifying process executed by a patient information providing system 1 is described with reference to FIG. 5 and FIG. 6. FIG. 5 and FIG. 6 are flowcharts showing an image specifying process executed by a server 10 and an information terminal 100. The processing executed by the above-described modules of each device is described together with this processing.

An image capturing module 170 captures a captured image of a patient (step S20). The processing in step S20 is the same as the processing in step S10 described above.

A position information acquiring module 171 acquires position information of an image capturing location (step S21). In step S21, the position information acquiring module 171 acquires the position information of the image capturing location by acquiring its own position information from a GPS or the like. Further, the position information acquiring module 171 may be configured to acquire its own position information by a sensor or the like, or may be configured to acquire its own position information by a configuration other than the GPS.

An information storage module 160 stores the acquired position information (step S22). In step S22, the information storage module 160 stores the position information and a current date and time in association with each other. The information storage module 160 may be configured to further store the position information and information other than the current date and time in association with each other, and may be configured to store only the position information.

Further, the information terminal 100 may be configured to execute the processing in step S21 and step S22 simultaneously with the processing in step S20. That is, when the image capturing module 170 captures the patient, the position information acquiring module 171 may acquire the position information and the information storage module 160 may store the position information.

An information transmitting/receiving module 150 transmits captured image information which is data of the captured image to the server 10 (step S23). Further, the information transmitting/receiving module 150 may be configured to execute the processing in step S23 prior to the processing in step S22 described above.

An information acquiring module 20 acquires the captured image information. An image analyzing module 40 performs image analysis on the captured image based on the captured image information, thereby specifying an ID of the patient appearing in the captured image (step S24). In step S24, the image analyzing module 40 extracts a feature amount of the patient by the image analysis. The image analyzing module 40 performs the image analysis on captured images based on captured image information stored in a patient ID database and extracts feature amounts. The image analyzing module 40 compares the feature amount of the patient extracted from the acquired captured image with the feature amounts extracted from the stored captured images, and specifies the ID associated with the matched captured image information.

When the ID has not been specified, that is, when the stored captured image having the feature amount which matches the feature amount of the patient extracted from the acquired captured image does not exist, the server 10 may be configured to notify a notification indicating this fact to the information terminal 100. In this case, for example, the information terminal 100 captures the patient at different angles based on this notification, and transmits the captured images of the respective angles as one captured image to the server 10. The server 10 acquires the one captured image and extracts a feature amount of the patient at each angle. The server 10 may be configured to specify an ID of the patient again based on the extracted feature amount. When the server 10 has not specified the ID even if repeating this process a predetermined number of times, the server 10 may be configured to accept an input of an ID or other necessary information in the information terminal 100, or to display precautions for capturing the patient in the information terminal 100 thereby prompting the medical staff to capturing the patient again.

A patient information specifying module 31 accesses a patient information database stored in an information storage module 30 to acquire patient information associated with the specified ID of the patient (step S25). In step S25, the patient information database is a database in which various information of patients to be stored as an electronic medical record are associated with IDs of the patients. For example, the patient information database may be configured based on patient information and IDs inputted by the medical staff from the information terminal 100, or may be configured based on patient information and IDs inputted from another terminal device or the like. The patient information is, for example, a name, an age, an occupation, a sex, an insurer number, an injury/illness name, a visit start date, a hospital stay period, a past medical history, a medication content, a surgical content, a remark, or the like.

In step S25, when accessing the patient information database, the patient information specifying module 31 may use the captured image information as an evidence for identification of the patient. In this case, when the patient or a related person such as a family member of the patient appears in the captured image, the patient information specifying module 31 permits access to the above-described various databases and specifies the patient information. When the patient or the related person such as the family member of the patient does not appear in the captured image, the patient information specifying module 31 denies the access to the above-described various databases and does not specify the patient information. Further, the patient information specifying module 31 may be configured to permit the access to the above-described various databases when only the patient appears in the captured image, and deny the access to the above-described various databases when the patient does not appear in the captured image.

An information transmission module 21 transmits the acquired patient information to the information terminal 100 (step S26).

An information transmitting/receiving module 150 receives the patient information. A display module 172 displays the received patient information (step S27). The display module 172 may be configured to display a part or all of the patient information, may be configured to display a change in the patient information as a graph, or may be configured to display position information of an image capturing location stored in an information storage module 160 and the patient information in association with each other. In addition, the display module 172 may display the above-described configurations in combination, or may use other display manners. The part of the patient information is, for example, information necessary for the patient's treatment, such as the patient's name, vital signs, medication information, or the like. Further, the patient information displayed by the display module 172 can be appropriately changed. Furthermore, the change in patient information is, for example, a change in the date and time of vital signs, a change in a case of a disease, a change in behavioral contents of one day, or the like.

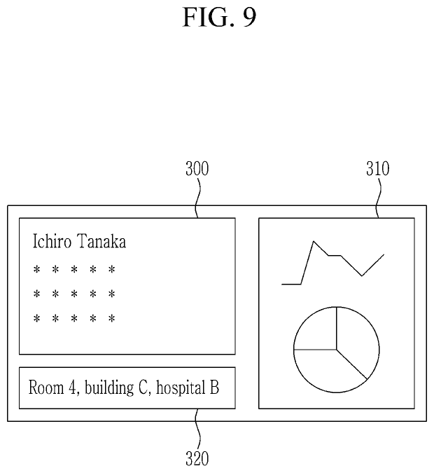

The patient information displayed by the display module 172 is described with reference to FIG. 9. FIG. 9 is a diagram showing an example of the patient information displayed by the display module 172. In FIG. 9, the display module 172 displays a patient information display area 300, a graph display area 310, and a position information display area 320 on its display unit. The patient information display area 300 is an area for displaying the patient information. The patient information displayed in the patient information display area 300 is, for example, a name of a patient, a past medical history, a case of a disease, an operation day, or the like. The patient information displayed in the patient information display area 300 is a part or all of the received patient information. In a case of displaying only the part, for example, a configuration in which only the patient information which is designated in advance by a medical staff is displayed, a configuration in which the information terminal 100 determines and displays necessary patient information based on a current date and time or a current location, a configuration in which the patient information to be displayed is designated based on an input operation such as a voice input or a gesture input and the designated patient information is displayed may be used. The graph display area 310 is an area for displaying a change in the patient information as a graph. The change in the patient information displayed in the graph display area 310 has, for example, a configuration in which a change in vital signs is displayed as a bar graph for each predetermined date and time, each a predetermined time, or the like, a configuration in which a time zone of rehabilitation, meals, bedtime, surgery, or the like is displayed as a pie chart, or a configuration in which a change in dosage is displayed as a line graph. The change in the patient information displayed in the graph display area 310 may have a configuration in which the change in the patient information which is designated in advance by the medical staff is displayed or a configuration in which patient information to be displayed as the graph is designated based on an input operation such as a voice input or a gesture input and the change in the designated patient information is displayed. Further, the position information display area 320 is an area which displays position information of an image capturing location. The position information displayed in the position information display area 320 is, for example, a name, address, coordinates, identifier or the like of a location based on the position information. The position information display area 320 is an area which displays position information of a location where an information code is read in a code reading process to be described below.

On the other hand, all of the patient information display area 300, the graph display area 310, and the position information display area 320 displayed by the display module 172 may not always need to be displayed, and any or a combination of them may be displayed. Further, the patient information to be displayed in the patient information display area 300 can be appropriately changed. Furthermore, a type or content of the graph displayed in the graph display area 310 can be appropriately changed. In addition, a content of the position information displayed in the position information display area 320 can be appropriately changed. Additionally, the display module 172 may be configured to display information other than the above information. For example, the display module 172 may be configured to display a captured image or may be configured to display an information code to be described below.

Further, the display module 172 may be configured to initially display the patient information display area 300, and then to receive an input operation such as a voice input or a gesture input from a medical staff and display the graph display area 310 or the position information display area 320. In other words, the display module 172 can freely select and display each of the above-described areas, and can also display a desired area at a desired timing.

The image capturing module 170 determines whether a capturing input of a captured image is accepted again (step S28). In step S28, when determining that the capturing input is not accepted (NO in step S28), the image capturing module 170 ends the present process.

On the other hand, when determining in step S28 that the capturing input is accepted (YES in step S28), the image capturing module 170 captures a captured image of the patient (step S29). The processing in step S29 is the same as the processing in step S20 described above.

The position information acquiring module 171 acquires its own position information (step S30). The processing in step S30 is the same as the processing in step S21 described above.

A location determining module 173 determines whether location information acquired at this time is approximate to location information stored in the information storage module 160 (step S31). Being approximate to the position information means, for example, whether the position information is in a range of the same room, whether both of the position information match, or the like. In step S31, when determining that the acquired location information is not approximate to the stored one (NO in step S31), the location determining module 173 executes the above-described processing in step S22.

In step S31, the location determining module 173 may be configured to determine whether the acquired location information is approximate to the stored one based on other criteria. Further, a range of the approximation can be appropriately changed.

On the other hand, when determining in step S31 that the acquired location information is approximate to the stored one (YES in step S31), a date and time determining module 174 determines whether a current date and time is approximate to the date and time stored in the information storage module 160 (step S32). Being approximate to the date and time means, for example, that a time difference between the current date and time and the stored date and time is within 10 minutes or within 1 hour, that the current date and time is the same as the stored date and time, or the like. In step S32, when determining that the current date and time is not approximate to the stored one (NO in step S32), the date and time determining module 174 executes the above-described processing in step S22.

In step S32, the date and time determining module 174 may be configured to determine whether the current date and time is approximate to the stored one based on other criteria. Further, the range of the approximation can be appropriately changed.

On the other hand, when determining in step S32 that the current date and time is approximate to the stored one (YES in step S32), the display module 172 displays the patient information displayed in step S27 again (step S33).

Further, the order of step S31 and step S32 described above may be reversed, or only one of them may be executed.

Through the processing in steps S28 to S33, in a case where the information terminal 100 once captures the patient and specifies the patient information of the patient, and thereafter captures the patient again, the information terminal 100 can display the patient information on assumption that the patient captured at this time is the patient with the ID associated with this patient information without executing the processing by the server 10 when they are within a predetermined location and a predetermined time. Further, the information terminal 100 can obtain the same effect even when both they are within the predetermined time or the predetermined time.

The above is the image specifying process.

In the image specifying process described above, the processing executed by the server 10 or the information terminal 100 may be configured to be collaboratively executed by the server 10 and the information terminal 100. Alternatively, a part or all of the processing executed by the server 10 may be configured to be executed by the information terminal 100, or a part or all of the processing executed by the information terminal 100 may be configured to be executed by the server 10.

Code Reading Process

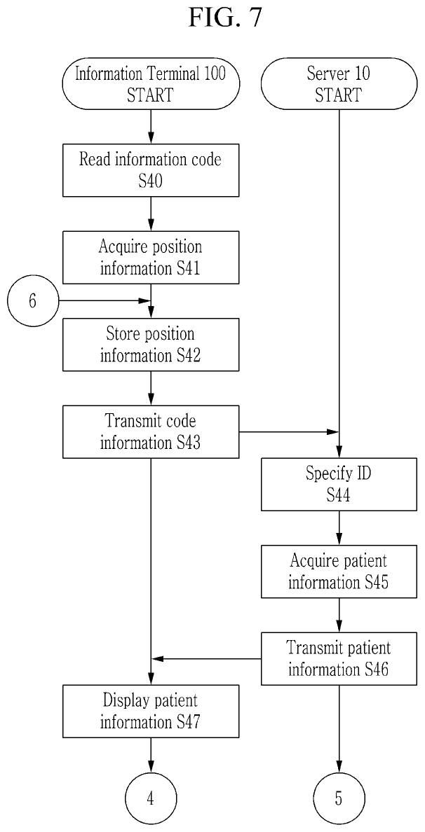

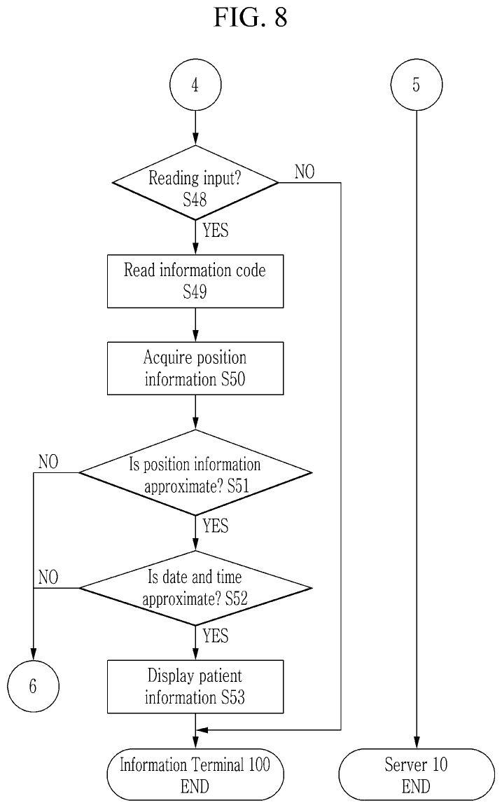

A code reading process executed by a patient information providing system 1 is described with reference to FIG. 7 and FIG. 8. FIG. 7 and FIG. 8 are flowcharts showing a code reading process executed by a server 10 and an information terminal 100. The processing executed by the above-described modules of each device is described together with this processing.

A reading module 175 reads an information code such as a two-dimensional code or a one-dimensional code of a patient (step S40). In step S40, the reading module 175 reads the information code attached to a skin of the patient or the information code attached to clothing or the like, by a dedicated application, an image capturing device, or the like. In step S40, the information code read by the reading module 175 is not limited to the above-described configuration, and can be appropriately changed. Further, the information code need not be attached to the patient himself/herself, the clothing, or the like, but may be provided in a place such as a bed or a door of a hospital room, other articles, or the like.

A position information acquiring module 171 acquires position information of a reading location (step S41). The processing in step S41 is the same as the processing in step S21 described above.

An information storage module 160 stores the position information acquired by the position information acquiring module 171 in step S41 (step S42). The processing in step S42 is the same as the processing in step S22 described above.

Further, the information terminal 100 may be configured to execute the processing in step S41 and step S42 simultaneously with the processing in step S40. In other words, when the reading module 175 reads the information code, the position information acquiring module 171 may acquire the position information and the information storing module 160 may store the position information.

An information transmitting/receiving module 150 transmits the code information which is data of the information code to the server 10 (step S43). Further, the information transmitting/receiving module 150 may be configured to execute the processing in step S43 prior to the processing in step S42 described above.

An information acquiring module 20 acquires the code information. Based on the code information, a code analyzing module 41 specifies an ID of the patient associated with this code information (step S44). The code analyzing module 41 specifies the ID of the patient by extracting the ID stored in the code information based on the code information.

A patient information specifying module 31 accesses a patient information database stored in an information storage module 30 to acquire the patient information associated with the specified ID of the patient (step S45). The processing in step S45 is the same as the processing in step S25 described above.

In step S45, when the patient information specifying module 31 accesses the patient information database, the code information may be used as an evidence for identification of the patient. In this case, when the ID is stored in the information code which is read, the patient information specifying module 31 permits access to the above-described various databases and specifies the patient information. When the ID is not stored in the information code which is read, the patient information specifying module 31 denies the access to the various databases and does not specify the patient information.

An information transmission module 21 transmits the acquired patient information to the information terminal 100 (step S46).

An information transmitting/receiving module 150 receives the patient information. A display module 172 displays the received patient information (step S47). The processing in step S47 is the same as the processing in step S27 described above. However, in step S47, the display module 172 does not display the position information of the image capturing location but displays the position information of a location where the information code is read.

The reading module 175 determines whether a reading input of an information code is accepted again (step S48). In step S48, when determining that the reading input is not accepted (NO in step S48), the reading module 175 ends the present process.

On the other hand, when determining in step S48 that the reading input is accepted (YES in step S48), the reading module 175 reads the information code (step S49). The processing in step S49 is the same as the processing in step S40 described above.

The position information acquiring module 171 acquires its own position information (step S50). The processing in step S50 is the same as the processing in step S41 described above.

The location determining module 173 determines whether the location information acquired at this time is approximate to the location information stored in the information storage module 160 (step S51). Being approximate to the position information is the same as the configuration of the processing in step S31 described above. In step S51, when determining that the acquired location information is not approximate to the stored one (NO in step S51), the location determining module 173 executes the above-described processing in the step S42.

In step S51, the location determining module 173 may be configured to determine whether the acquired location information is approximate to the stored one based on other criteria. Further, the range of the approximation can be appropriately changed.

On the other hand, when determining in step S51 that the acquired location information is approximate to the stored one (YES in step S51), the date and time determining module 174 determines whether the current date and time is approximate to the date and time stored in the information storage module 160 (Step S52). Being approximate to the date and time is the same as the configuration of the processing in step S32 described above. In step S52, when determining that the current date and time is not approximate to the stored one (step S52 NO), the date and time determining module 174 executes the above-described processing in step S42.

In step S52, the date and time determining module 174 may be configured to determine whether the current date and time is approximate to the stored one based on other criteria. Further, the range of the approximation can be appropriately changed.

On the other hand, when determining in step S52 that the current date and time is approximate to the stored one (YES in step S52), the display module 172 again displays the patient information displayed in step S47 described above (step S53).

Further, the order of steps S51 and S52 described above may be reversed, or only one of them may be executed.

Through the processing in steps S48 to 533, in a case where the information terminal 100 once reads the information code of the patient and specifies the patient information of the patient, and thereafter reads the information code of the patient again, the information terminal 100 can display the patient information on assumption that the patient having the information code read at this time is the patient with the ID associated with this patient information without executing the processing by the server 10 when they are within a predetermined location and a predetermined time. Further, the information terminal 100 can obtain the same effect even when they are within the predetermined time or the predetermined time.

The above is the code reading process.

In the above-described code reading process, the processing executed by the server 10 or the information terminal 100 may be configured to be collaboratively executed by the server 10 and the information terminal 100. Alternatively, a part or the whole of the processing executed by the server 10 may be configured to be executed by the information terminal 100, or a part or the whole of the processing executed by the information terminal 100 may be configured to be executed by the server 10.

In the above-described embodiments, the image specifying process and the code reading process are performed independently, but a configuration in which both are executed in combination may be adopted. For example, it may be configured to capture the captured image of the patient and read the information code. By doing so, the specification of the patient is executed by the captured image and the information code, so that the specification of the patient can be more reliably executed.

The means and functions described above are realized by reading and executing a predetermined program by a computer (including a CPU, an information processing device, or various terminals). The program is provided, for example, in a form recorded in a computer-readable recording medium such as a flexible disk, a CD (e.g., CD-ROM or the like), a DVD (DVD-ROM, DVD-RAM, or the like), or the like. In this case, the computer reads the program from the recording medium and transfers the program to an internal storage unit or an external storage unit so as to be stored and executed. Furthermore, the program may be, for example, recorded in a storage device (recording medium) such as a magnetic disk, an optical disk, an optical magnetic disk, or the like in advance and be provided from the recording medium to the computer via communication line.

While the embodiments of the present invention have been described above, the present invention is not limited to the above-described embodiments. In addition, the effects described in the embodiments of the present invention are merely a list of the most preferable effects produced by the present invention, and the effects of the present invention are limited to those described in the embodiments of the present invention.

### DESCRIPTION OF REFERENCE NUMBERS

1: patient information providing system, 10: server, 100: information terminal

1. A patient information providing system for accessing a storage unit that stores patient information to acquire and display the patient information, the patient information providing system comprising:
a processor that:
acquires a first captured image captured by a terminal;
acquires position information of a location where the first captured image is captured by the terminal;
acquires a date and time when the first captured image is captured by the terminal;
specifies an ID of a patient appearing in the acquired first captured image by image recognition;
acquires patient information associated with the specified ID of the patient from the storage unit;
displays the acquired patient information on a display unit;
acquires a second captured image captured by the terminal again;
determines whether position information of a location where the second capture image is captured by the terminal is approximate to the position information, which is previously acquired, of the location where the first capture image is captured;
determines whether a date and time when the second capture image is captured by the terminal is approximate to the date and time, which is previously acquired, when the first captured image is captured; and
displays on the display unit the previously acquired patient information again without specifying the ID of the patient and without acquiring the patient information from the storage unit when the position information of the location where the second capture image is captured and the position information which is previously acquired are approximate and when the date and time when the second capture image is captured and the date and time which is previously acquired are approximate.

2. A patient information providing system for accessing a storage unit that stores patient information to acquire and display the patient information, the patient information providing system comprising:
a processor that:
acquires a first two-dimensional code read by a terminal;
acquires position information of a location where the first two-dimensional code is read by the terminal;
acquires a date and time when the first two-dimensional code is read by the terminal;
specifies an ID of a patient associated with the acquired first two-dimensional code;
acquires patient information associated with the specified ID of the patient from the storage unit;
displays the acquired patient information on a display unit;
acquires a second two-dimensional code read by the terminal again;
determines whether position information of a location where the second two-dimensional code is read by the terminal is approximate to the position information, which is previously acquired, of the location where the first two-dimensional code is read;
determines whether a date and time when the second two-dimensional code is read by the terminal is approximate to the date and time, which is previously acquired, when the first two-dimensional code is read; and
displays on the display unit the previously acquired patient information again without specifying the ID of the patient and without acquiring the patient information from the storage unit when the position information of the location where the second two-dimensional code is read and the position information which is previously acquired are approximate and when the date and time when the second two-dimensional code is read and the date and time which is previously acquired are approximate.

3. The patient information providing system according to claim 1, wherein the display unit displays a change in the acquired patient information as a graph.

4. The patient information providing system according to claim 1, wherein the display unit displays the acquired patient information in association with the acquired position information of the first captured image.

5. The patient information providing system according to claim 2, wherein the display unit displays the acquired patient information in association with the acquired position information of the first two-dimensional code.

6. The patient information providing system according to claim 1, wherein the processor uses the first captured image as an evidence of the specified patient when accessing the storage unit.

7. The patient information providing system according to claim 2, wherein the processor uses the first two-dimensional code as an evidence of the specified patient when accessing the storage unit.

8. A patient information providing method of accessing a storage device that stores patient information to acquire and display the patient information, the patient information providing method comprising:
acquiring a first captured image captured by a terminal;
acquiring position information of a location where the first captured image is captured by the terminal;
acquiring a date and time when the first captured image is captured by the terminal;
specifying an ID of a patient appearing in the acquired first captured image by image recognition;
acquiring patient information associated with the specified ID of the patient from the storage unit;
displaying the acquired patient information on a display unit;
acquiring a second captured image captured by the terminal again;
determining whether position information of a location where the second capture image is captured by the terminal is approximate to the position information, which is previously acquired, of the location where the first capture image is captured;
determining whether a date and time when the second capture image is captured by the terminal is approximate to the date and time, which is previously acquired, when the first captured image is captured; and
displaying on the display unit the previously acquired patient information again without specifying the ID of the patient and without acquiring the patient information from the storage unit when the position information of the location where the second capture image is captured and the position information which is previously acquired are approximate and when the date and time when the second capture image is captured and the date and time which is previously acquired are approximate.

9. A patient information providing method of accessing a storage unit that stores patient information to acquire and display the patient information, the patient information providing method comprising:
acquiring a first two-dimensional code read by a terminal;
acquiring position information of a location where the first two-dimensional code is read by the terminal;
acquiring a date and time when the first two-dimensional code is read by the terminal;
specifying an ID of a patient associated with the acquired first two-dimensional code;
acquiring patient information associated with the specified ID of the patient from the storage unit;
displaying the acquired patient information on a display unit;
acquiring a second two-dimensional code read by the terminal again;
determining whether position information of a location where the second two-dimensional code is read by the terminal is approximate to the position information, which is previously acquired, of the location where the first two-dimensional code is read;
determining whether a date and time when the second two-dimensional code is read by the terminal is approximate to the date and time, which is previously acquired, when the first two-dimensional code is read; and
displaying on the display unit the previously acquired patient information again without specifying the ID of the patient and without acquiring the patient information from the storage unit when the position information of the location where the second two-dimensional code is read and the position information which is previously acquired are approximate and when the date and time when the second two-dimensional code is read and the date and time which is previously acquired times when the first and second two dimensional codes are read are approximate.

10. A program for causing a patient information providing system accessing a storage unit that stores patient information to acquire and display the patient information to execute:
acquiring a first captured image captured by a terminal;
acquiring position information of a location where the first captured image is captured by the terminal;
acquiring a date and time when the first captured image is captured by the terminal;
specifying an ID of a patient appearing in the acquired first captured image by image recognition;
acquiring patient information associated with the specified ID of the patient from the storage unit;
displaying the acquired patient information on a display unit;
acquiring a second captured image captured by the terminal again;
determining whether position information of a location where the second capture image is captured by the terminal is approximate to the position information, which is previously acquired, of the location where the first capture image is captured;
determining whether a date and time when the second capture image is captured by the terminal is approximate to the date and time, which is previously acquired, when the first captured image is captured; and
displaying on the display unit the previously acquired patient information again without specifying the ID of the patient and without acquiring the patient information from the storage unit when the position information of the location where the second capture image is captured and the position information which is previously acquired are approximate and when the date and time when the second capture image is captured and the date and time which is previously acquired are approximate.

11. A program for causing a patient information providing system accessing a storage unit that stores patient information to acquire and display the patient information to execute:
acquiring a first two-dimensional code read by a terminal;
acquiring position information of a location where the first two-dimensional code is read by the terminal;
acquiring a date and time when the first two-dimensional code is read by the terminal;
specifying an ID of a patient associated with the acquired first two-dimensional code;
acquiring patient information associated with the specified ID of the patient from the storage unit;
displaying the acquired patient information on a display unit;
acquiring a second two-dimensional code read by the terminal again;
determining whether position information of a location where the second two-dimensional code is read by the terminal is approximate to the position information, which is previously acquired, of the location where the first two-dimensional code is read;
determining whether a date and time when the second two-dimensional code is read by the terminal is approximate to the date and time, which is previously acquired, when the first two-dimensional code is read; and
displaying on the display unit the previously acquired patient information again without specifying the ID of the patient and without acquiring the patient information from the storage unit when the position information of the location where the second two-dimensional code is read and the position information which is previously acquired are approximate and when the date and time when the second two-dimensional code is read and the date and time which is previously acquired are approximate.

12. The patient information providing system according to claim 2, wherein the display unit displays a change in the acquired patient information as a graph.
